(19) United States
(12) Patent Application Publication
(10) Pub. No.: US 2026/0365782 A1
(43) Pub. Date: Nov. 19, 2026

(54) MODIFIED CURING AGENT COMPONENT AND A PROCESS FOR PREPARING THE SAME

(71) Applicant: Grasim Industries Limited, Nagda, Madhya Pradesh (IN)

(72) Inventors: Mukesh Shyamrao KATHALEWAR, Panvel (IN); Diganta KHATANIAR, Panvel (IN); Bharat SINGH, Panvel (IN); Pradip Kumar DUBEY, Mumbai (IN)

(73) Assignee: Grasim Industries Limited, Nagda, Madhya Pradesh (IN)

(21) Appl. No.: 19/478,289

(22) PCT Filed: Apr. 24, 2024
(86) PCT No.: PCT/IB2024/053989

§ 371 Date: Oct. 24, 2025

(30) Foreign Application Priority Data
Apr. 24, 2023 (IN) 202321029571

(51) Int. Cl. C08G 59/50; C07C 213/08; C08G 59/62; C08L 63/00

(52) U.S. Cl. CPC C08G 59/504; C07C 213/08; C08G 59/623; C08L 63/00

(57) ABSTRACT

A modified curing agent component and a process for preparing said modified curing agent component is disclosed. The modified curing agent component comprises a compound of Formula I, a compound of Formula II and a compound of Formula III. A recyclable epoxy resin system comprising at least one epoxy resin component and the modified curing agent component is disclosed. The disclosed recyclable epoxy resin system has a curing temperature in the range of 0 °C-10 °C.

### FIELD OF THE INVENTION

The present disclosure relates to a modified curing agent component and a process for preparing said modified curing agent component.

### BACKGROUND

Epoxy resins are class of polymer materials used extensively in diverse applications like performance materials, structural materials, aerospace composites, automobile composites, windmill composites, adhesives, paints, coatings, electrical, electronics, printed circuit boards PCBs, moulding, potting and other specialty composites applications. Epoxy resins offer superior mechanical and thermal properties such as high strength, high chemical and solvent resistance, low shrinkage, excellent adhesion to varying substrates, economical with low toxicity.

To prepare epoxy resin compositions, the epoxy resins, also known as the epoxy resin component, are required to be cured using a curing agent. Curing is the process whereby the liquid epoxy resin component is transformed into a solid, durable material. During curing, the epoxy resin component reacts with a curing agent to form a three-dimensional cross-linked thermoset structure. Known curing agents include phenols, anhydrides, and amines. Curing agents often affect the properties of the cured epoxy resin compositions.

Thus, it would be desirable to have a curing agent for epoxy resins that gives a balance of properties to the cured epoxy resins.

### SUMMARY

A modified curing agent component for an epoxy resin system is disclosed. The modified curing agent component comprises of:
a compound of Formula I represented by:

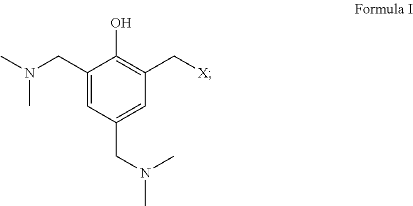

wherein each of X is independently selected from:

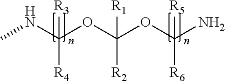

wherein:
n is 1-5; and
each of R₁, R₂, R₃, R₄, R₅ and R₆ is selected from the group consisting of H, CH₃, C₂H₅ and a combination thereof;
or

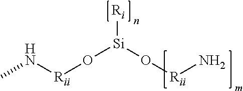

wherein
n is 0-2;
m is 1-3;
Rᵢ is independently methyl, or ethyl; and each of Rᵢᵢ is independently selected from the group consisting of ethylene, propylene, isopropylene, butylene, isobutylene and a combination thereof;
   a compound of Formula II represented by:

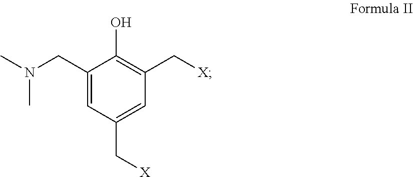

wherein each of X is independently selected from:

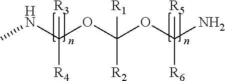

wherein:
n is 1-5; and each of R1, R2, R3, R4, R5 and R6 is independently selected from the group consisting of H, CH3, C2H5 and a combination thereof;
or

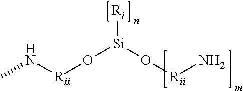

wherein
n is 0-2;
m is 1-3;
Ri is independently methyl, or ethyl; and
each of Rᵢᵢ is independently selected from the group consisting of ethylene, propylene, isopropylene, butylene, isobutylene and a combination thereof; and
   a compound of Formula III, represented by:

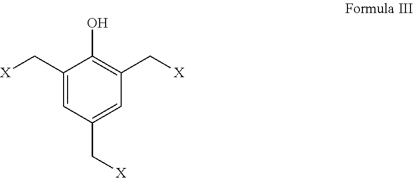

wherein each of X is independently selected from:

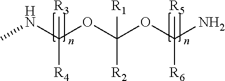

wherein:
n is 1-5; and
each of R1, R2, R3, R4, R5 and R6 is independently selected from the group consisting of H, CH3, C2H5 and a combination thereof;
or

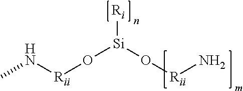

wherein
n is 0-2;
m is 1-3;
Rᵢ is independently selected from methyl, or ethyl; and
each of Rᵢᵢ is independently selected from the group consisting of ethylene, propylene, isopropylene, butylene, isobutylene and a combination thereof.

The process for preparing a modified curing agent component is also disclosed. Said process comprising reacting a compound of Formula IV with a compound selected from the group consisting of a compound of Formula V, a compound of Formula VI and a combination thereof, wherein the compound of Formula IV, the compound of Formula V and the compound of Formula VI are represented by the structures:

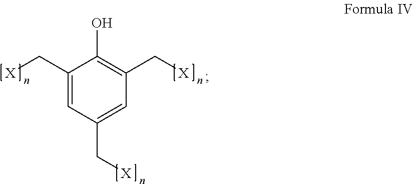

wherein
n is 0-1;
X is either

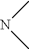

or OH;

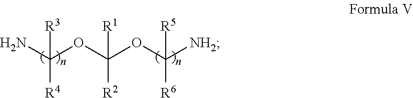

wherein
n is 1-5;
each R₁ to R₆ is independently selected from the group consisting of H, CH₃, and C₂H₅; and

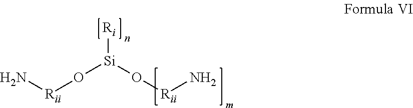

wherein
n is 0-2;
m is 1-3;
Rᵢ is independently selected from methyl, or ethyl;
each of Rᵢᵢ is independently selected from the group consisting of ethylene, propylene, isopropylene, butylene, isobutylene and a combination thereof; to obtain the aforementioned modified curing agent component.

A recyclable epoxy resin system comprising at least one epoxy resin component and the aforementioned modified curing agent component is disclosed. The disclosed recyclable epoxy resin system has a curing temperature in the range of 0 °C-10 °C.

### DETAILED DESCRIPTION OF THE INVENTION

For the purpose of promoting an understanding of the principles of the invention, reference will now be made to embodiments and specific language will be used to describe the same. It will nevertheless be understood that no limitation of the scope of the invention is thereby intended, such alterations and further modifications in the disclosed process, and such further applications of the principles of the invention therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “one embodiment” “an embodiment” or similar language means that a particular feature, structure, or characteristic described in connection with the embodiment is included in at least one embodiment of the present invention. Thus, appearances of the phrase “in one embodiment”, “in an embodiment” and similar language throughout this specification may, but do not necessarily, all refer to the same embodiment.

“Glass transition temperature” means the temperature range at which the cured epoxy resin changes from a hard, rigid or a “glassy” state to a “rubbery” state. The unit of the glass transition temperature is °C.

“Gel time” is defined as the amount of time it takes for a mixture of an epoxy resin component and curing agent component to gel completely. The unit of gel time is minutes.

“Curing temperature” is defined as the temperature at which the epoxy resin component cures with the curing agent component to form a three-dimensional cross-linked thermoset structure.

“Shore-D hardness” is a test method to determine the hardness of a material. The Shore D hardness rating is determined by how far an indenter can go into a material when a specified force is applied.

In the broadest scope, a modified curing agent component for an epoxy resin system, that allows said system to be recyclable is disclosed. The disclosed modified curing agent component rapidly reacts with an epoxy resin component both at room temperature and at a low temperature. The modified curing agent component thus enables the epoxy resin system to achieve the desired performance properties even at the low temperature. In an embodiment, the modified curing agent component cures with the epoxy resin component at a temperature in the range of 0-10 °C.

In one aspect, a modified curing agent component for an epoxy resin system is disclosed. The modified curing agent component comprising of:
a compound of Formula I represented by:

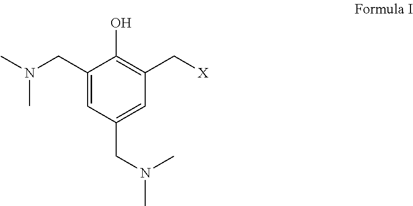

wherein each of X is independently selected from:

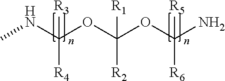

wherein n is 1-5; and
each of R₁, R₂, R₃, R₄, R₅ and R₆ is selected from the group consisting of H, CH₃, C₂H₅ and a combination thereof;
or

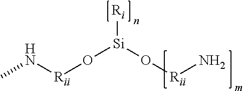

wherein
n is 0-2;
m is 1-3;
Rᵢ is independently selected from methyl, or ethyl;
each of Rᵢᵢ is independently selected from the group consisting of ethylene, propylene, isopropylene, butylene, isobutylene and a combination thereof;
   a compound of Formula II represented by:

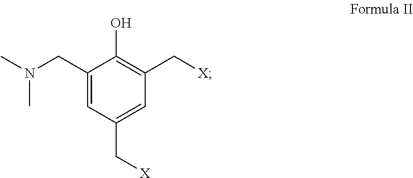

wherein each of X is independently selected from:

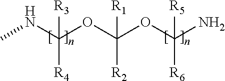

wherein n is 1-5; and
each of R₁, R₂, R₃, R₄, R₅ and R₆ is selected from the group consisting of H, CH₃, C₂H₅ and a combination thereof;
or

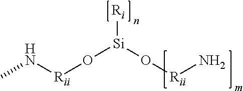

wherein
n is 0-2;
m is 1-3;
Rᵢ is independently methyl, or ethyl; and
each of Rᵢᵢ is independently selected from the group consisting of ethylene, propylene, isopropylene, butylene, isobutylene and a combination thereof; and
   a compound of Formula III, represented by:

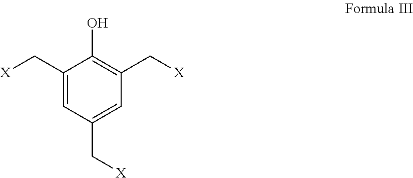

wherein each of X is independently selected from:

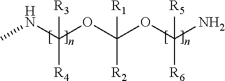

wherein n is 1-5; and
each of R₁, R₂, R₃, R₄, R₅ and R₆ is independently selected from the group consisting of H, CH₃, C₂H₅ and a combination thereof;
or

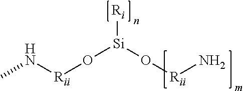

wherein
n is 0-2;
m is 1-3;
Rᵢ is independently selected from methyl, or ethyl; and
each of Rᵢᵢ is independently selected from the group consisting of ethylene, propylene, isopropylene, butylene, isobutylene and a combination thereof.

In an embodiment, the compound of Formula I is selected from the group consisting of a compound of Formula IA, a compound of Formula IB, a compound of Formula IC and a compound of Formula ID represented by:

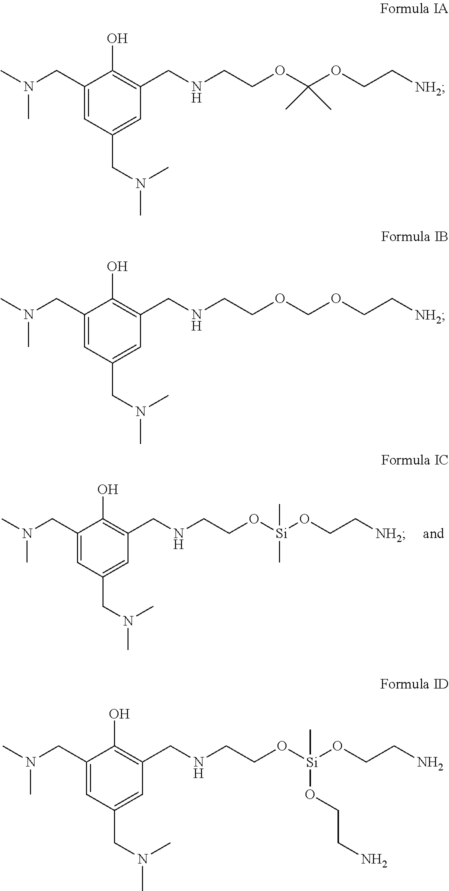 [SMILES: CN(C)Cc1cc(CNCCOC(C)(C)OCCN)c(O)c(CN(C)C)c1 ; CN(C)Cc1cc(CNCCOCOCCN)c(O)c(CN(C)C)c1 ; CN(C)Cc1cc(CNCCO[Si](C)(C)OCCN)c(O)c(CN(C)C)c1 ; CN(C)Cc1cc(CNCCO[Si](C)(OCCN)OCCN)c(O)c(CN(C)C)c1]

In some embodiments, the compound of Formula (I) is Formula IA.

In an embodiment, the compound of Formula II is selected from the group consisting of a compound of Formula IIA, a compound of Formula IIB, a compound of Formula IIC, and a compound of Formula IID represented by:

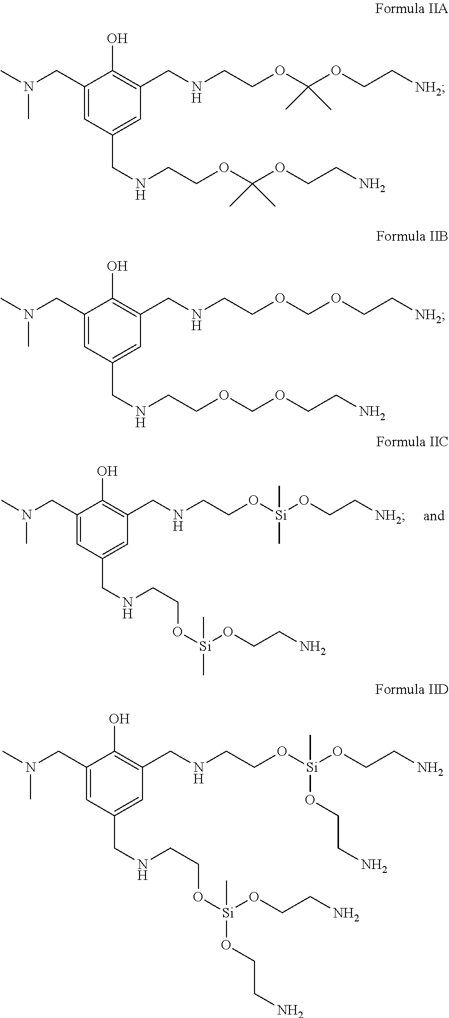 [SMILES: CN(C)Cc1cc(CNCCOC(C)(C)OCCN)cc(CNCCOC(C)(C)OCCN)c1O ; CN(C)Cc1cc(CNCCOCOCCN)cc(CNCCOCOCCN)c1O ; CN(C)Cc1cc(CNCCO[Si](C)(C)OCCN)cc(CNCCO[Si](C)(C)OCCN)c1O ; CN(C)Cc1cc(CNCCO[Si](C)(OCCN)OCCN)cc(CNCCO[Si](C)(OCCN)OCCN)c1O]

In some embodiments, the compound of Formula II is the compound of Formula IIA.

In an embodiment, the compound of Formula III is selected from a group consisting of a compound of Formula IIIA, a compound of Formula IIIB, a compound of Formula IIIC, a compound of Formula HID, a compound of Formula IIIE and a compound of Formula IIIF represented by:

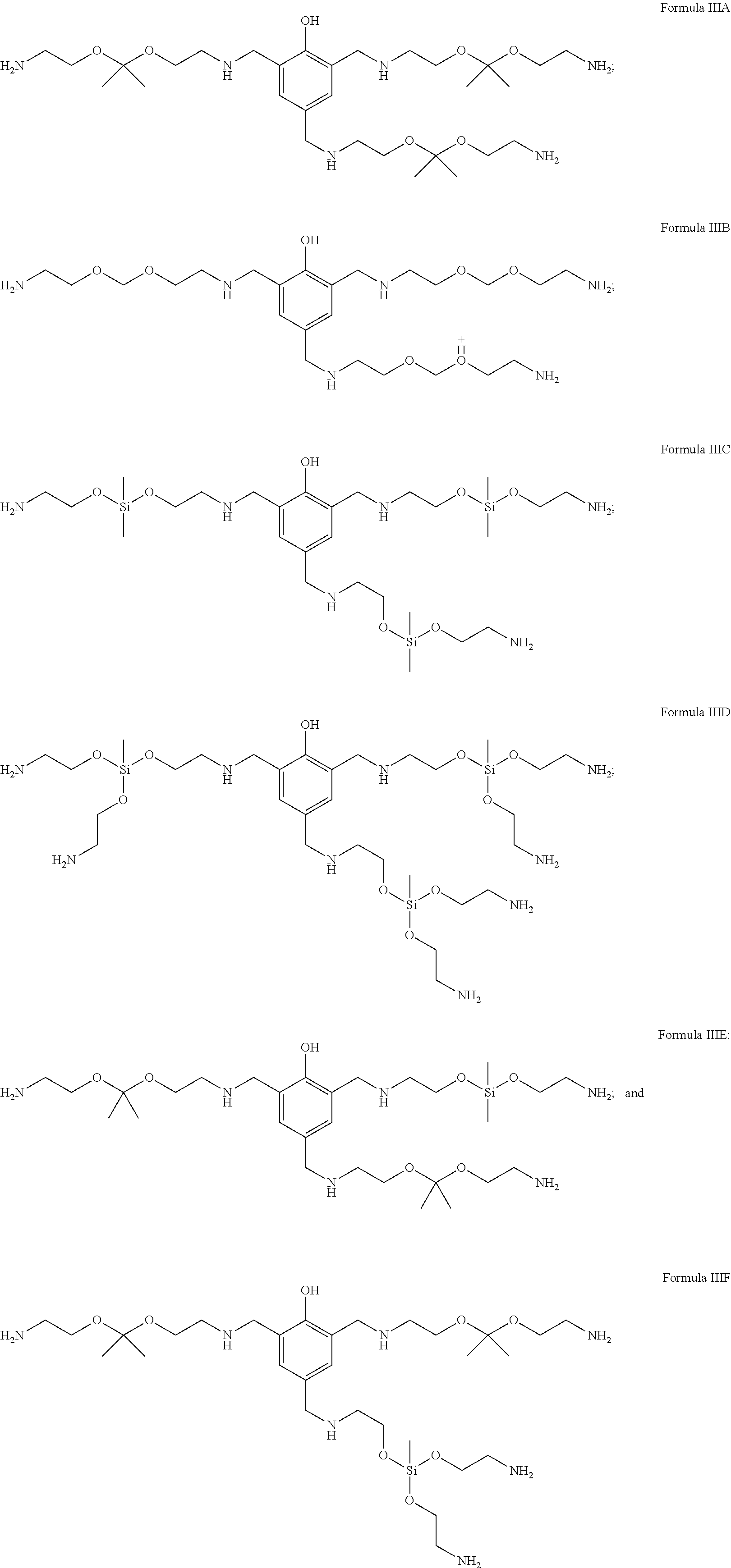

In some embodiments, the compound of Formula III is the compound of Formula IIIA represented by:

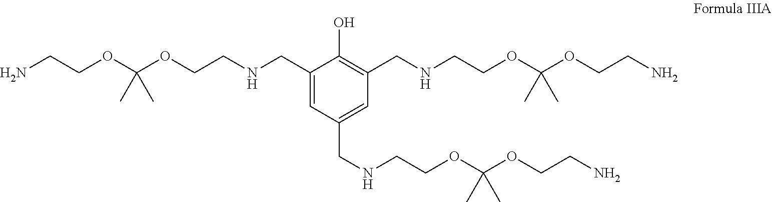 [SMILES: CC(C)(OCCN)OCCNCc1cc(CNCCOC(C)(C)OCCN)c(O)c(CNCCOC(C)(C)OCCN)c1]

In some embodiments, the modified curing agent component comprises the compound of Formula IA, the compound of Formula IIA, and the compound of Formula IIIA in variable weight proportion depending on a molar ratio of the starting materials used. In some embodiments, the modified curing agent component comprises the compound of Formula IA, the compound of Formula IIA, the compound of Formula IIIA, the compound of Formula IC, the compound of Formula IIC, the compound of Formula IIIC, and the compound of Formula HID in variable weight proportion depending on a molar ratio of the starting materials used.

In an embodiment, the modified curing agent component has a viscosity in the range of 200 to 80000 cPs at 25 °C. In some embodiments, the modified curing agent component has the viscosity in the range of 500 to 20000 cPs at 25 °C.

In an embodiment, the modified curing agent component has an amine value in the range of 300 to 800 mgKOH/gm. In some embodiments, the modified curing agent component has the amine value in the range of 350 to 650 mgKOH/gm.

In an embodiment, the modified curing agent component has an Amine hydrogen equivalent weight (AHEW) in the range of 50 to 250. In some embodiments, the modified curing agent component has the AHEW in the range of 65 to 180.

The modified curing agent component which is a mixture of the compound of Formula I, the compound of Formula II and the compound of Formula III, when used as a curing agent for curing an epoxy resin component, allows a cured epoxy resin thermoset based on these modified curing agent components to chemically breakdown to a low molecular weight thermoplastic material under required recycling conditions. The modified curing agent component as disclosed also results in improved performance properties such as higher glass transition temperature, mechanical strength and faster Shore-D hardness at low temperatures.

In accordance with an embodiment, the modified curing agent component comprising the compound of Formula I, the compound of Formula II and the compound of Formula III include either an acetal linkage, a ketal linkage, and a formal linkage. The acetal linkage, the ketal linkage, and the formal linkage of the modified curing agent component comprising the compound of Formula I, the compound of Formula II and the compound of Formula III are degradable upon exposure to an elevated temperature in an acid solution. In an embodiment, the degradation of the modified curing agent component is carried out at a temperature in the range of 60 to 100 °C. In an embodiment, the acid is selected from the group consisting of formic acid, acetic acid, and hydrochloric acid.

In an aspect, the compound of Formula I in the modified curing agent component is a monomeric compound, the compound of Formula II in the modified curing agent component is an oligomeric compound and the compound of Formula III in the modified curing agent component is an oligomeric compound that is a fully transaminated product. In as aspect, the higher amount of the transaminated compound of Formula III results in a higher proportion of the functional amine molecules within the product mixture which in turn results in increased viscosity of the modified curing agent component.

### Process of Preparing the Modified Curing Agent Component

In another aspect, a process for preparing the modified curing agent component is disclosed.

The modified curing agent component comprising the compound of Formula I, the compound of Formula II, and the compound of Formula III is obtained by the transamination reaction of a compound of Formula IV with a compound selected from the group consisting of a compound of Formula V, a compound of Formula VI and a combination thereof.

The compound of Formula IV, the compound of Formula V, and the compound of Formula VI are represented by the structures:

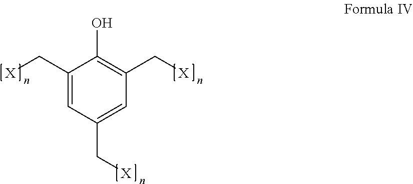

wherein X is either

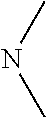

or OH; and
n is 0-1;

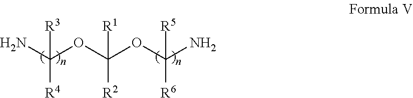

wherein n is 1 to 5; and
each R₁ to R₆ is independently selected from the group consisting of H, CH₃, and C₂H₅;
or

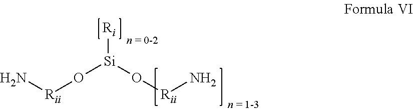

wherein Rᵢ is independently selected from methyl, or ethyl;
each of Rᵢᵢ is independently selected from the group consisting of ethylene, propylene, isopropylene, butylene, isobutylene and a combination thereof.

### Formula IV

In an embodiment, the compound of Formula IV is selected from the group consisting of tris(dimethylaminomethyl)phenol, bis[(dimethylamino)methyl]phenol, ((dimethylamino)methyl)phenol, and a combination thereof. In another embodiment, the compound of Formula IV is selected from the group consisting of 2,4-bihydroxymethyl phenol, 2,6-bishydroxymethyl phenol, and a combination thereof.

In some embodiments, the compound of Formula IV is Tris(dimethylaminomethyl)phenol.

### Formula V

The compound of Formula V is selected from the group consisting of a compound of Formula VA, a compound of Formula VB, a compound of Formula VC, a compound of Formula VD, and a compound of Formula VE represented by structures:

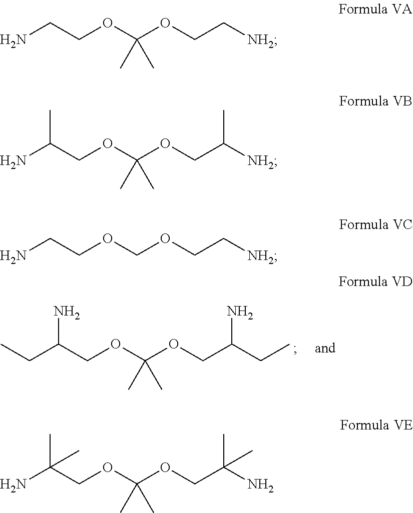

In some embodiments, the compound of Formula V is the compound of Formula VA and the compound of Formula VB.

### Formula VI

The compound of Formula VI is selected from the group consisting of a compound of Formula VIA, a compound of Formula VIB, a compound of Formula VIC, a compound of Formula VID, and a compound of Formula VIE represented by structures:

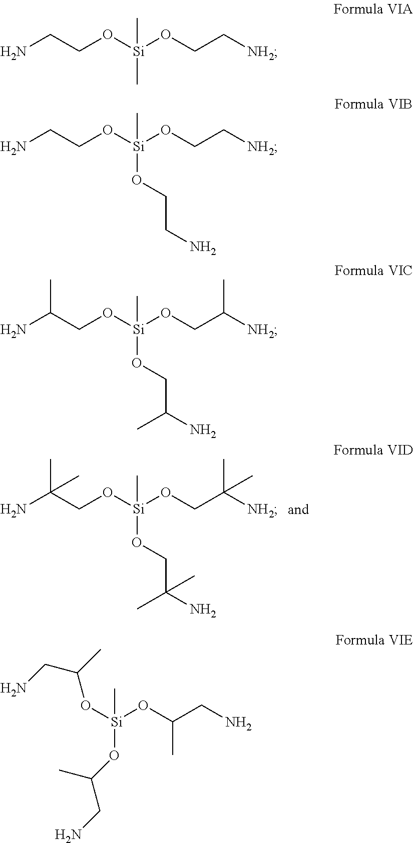

In some embodiments, the compound of Formula VI is the compound of Formula VIA. In the disclosed process, the molar ratio of each of the compound of Formula IV, the compound of Formula V, and the compound of Formula VI, along with the reaction temperature and the reaction time affects the extent of transamination and thereby the proportion of the compound of Formula I, the compound of Formula II and the compound of Formula III that are formed.

The reaction of the compound of Formula IV with the compound of Formula V or the compound of Formula VI or the combination thereof results in the formation of dimethylamine (DMA) gas. During this reaction, dimethylamine is liberated as a side product. The degree of reaction (% T) between the compound of Formula IV and the compound of Formula V or the compound of Formula VI or the combination thereof is determined by measuring the amount of the DMA liberated during the reaction. The weight loss of the reaction after the completion of the reaction is considered as the actual amount of the DMA liberated. The degree of transamination is calculated by using the following formula:

$$The degree of transamination ( \% T ) = \frac{Weight loss in the reaction * 100}{Theoretical amount of DMA liberated}$$

The DMA formed during the reaction of the compound of Formula IV with the compound of Formula V or the compound of Formula VI or the combination thereof is removed from the reaction mixture continuously and is streamed to a flask filled with water where it dissolves. The molar ratio of the compound of Formula IV, the compound of Formula V, the compound of Formula VI and the combination thereof used in the reaction along with the reaction temperature and the reaction time affects amount of the dimethylamine liberated.

### Formula IV and Formula V

In one embodiment, the process comprises reacting the compound of Formula IV with a compound of Formula V to obtain the modified curing agent component comprising the compound of Formula I, the compound of Formula II and the compound of Formula III. In some embodiments, the compound of Formula IV is reacted with the compound of Formula V in a molar ratio in the range of 1:2 to 1:6. In some embodiments, the compound of Formula IV is reacted with the compound of Formula V in a molar ratio in the range of 1:2.5 to 1:4.

In the reaction of the compound of Formula IV with Formula V, if an excess molar concentration of the compound of Formula V as compared to molar concentration of the compound of Formula IV is reacted, then greater amount of the compound of Formula I and the compound of Formula II are formed than the compound of Formula III. In an exemplary embodiment, when more than 3 moles of the compound of Formula V is reacted with 1 mole of the compound of Formula IV, then greater amount of compound of Formula I and Formula II are formed, as compared to the compound of Formula III. In another exemplary embodiment, when 2-3 moles of the compound of Formula V is reacted with 1 mole of compound of formula IV, then greater amount of compound of Formula III is formed as compared to the compound of Formula I and the compound of Formula II.

The reaction of the compound of Formula IV with the compound of Formula V is carried out at an elevated temperature. In an embodiment, the reaction of the compound of Formula IV with the compound of Formula V is carried out at a temperature in the range of 100-160 °C for a time-period in the range of 2 to 10 hours. In some embodiments, the reaction is carried out at 120-130 °C for 4 to 6 hours. If the reaction of the compound of Formula IV with the compound of Formula V is carried out for more than 240 minutes, it results in increase in extent of transamination and increase in viscosity.

An exemplary reaction scheme for obtaining the disclosed modified curing agent component (s) is provided below:

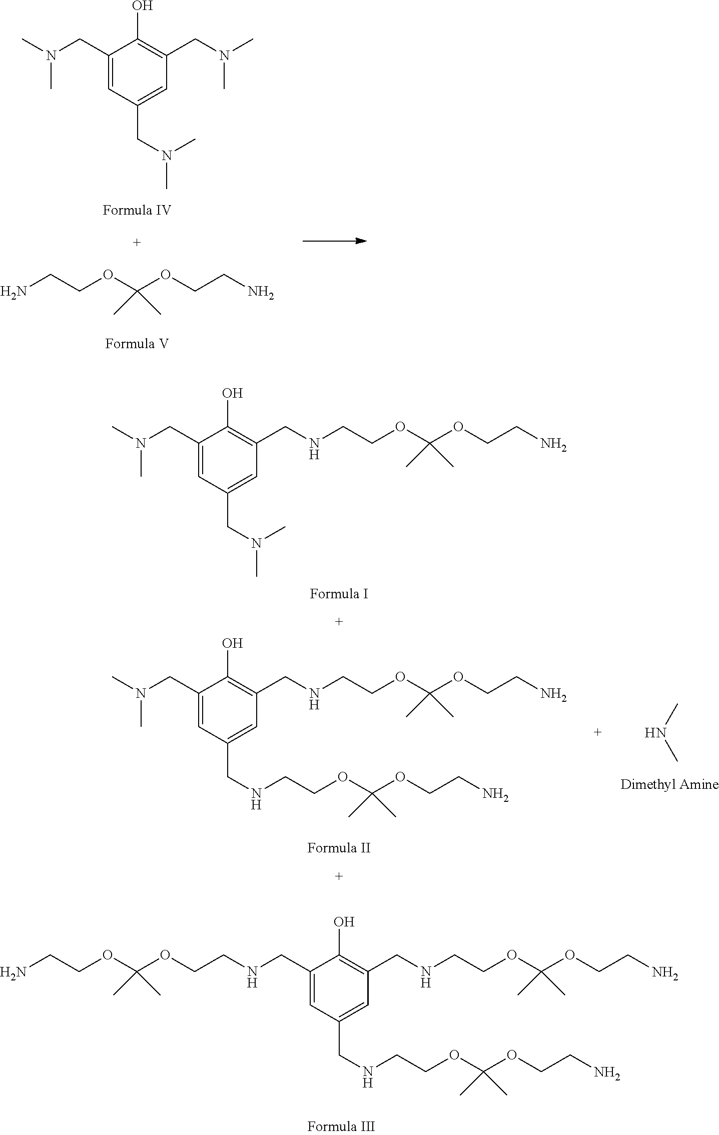

As shown above in the reaction scheme, the reaction of the compound of Formula IV with the compound of Formula V results in the formation of the modified curing agent component comprising the compound of Formula I, the compound of Formula II, and the compound of Formula III. The phenolic OH group present in the compound of Formula IV remains intact in the obtained modified curing agent component comprising the compound of Formula I, the compound of Formula II, and the compound of Formula III. The phenolic group catalyzes the reaction between the epoxy resin component and the modified curing agent component by activating epoxide ring of the epoxide resin component. The effect is prominent even at lower temperatures.

### Formula IV+Formula VI

In an embodiment, the compound of Formula IV is reacted with the compound of Formula VI in a molar ratio in the range of 1:2 to 1:6. In some embodiments, the compound of Formula IV is reacted with the compound of Formula VI in the molar ratio in the range of 1:2.5 to 1:4.

In the reaction of the compound of Formula IV with the compound of Formula VI, if an excess molar concentration of the compound of Formula VI as compared to the compound of Formula IV is reacted, then greater amount of the compound of Formula I and the compound of Formula II are formed as compared to the compound of Formula III. In an exemplary embodiment, when 3 moles of the compound of Formula VIB is reacted with 1 mole of the compound of Formula IV, then greater amount of compound of Formula ID and Formula IID are formed in comparison to the compound of Formula IIIF. In another exemplary embodiment, when 2-3 moles of the compound of Formula VI is reacted with 1 mole of the compound of Formula IV, then greater amount of the compound of Formula HID is formed as compared to the compound of Formula ID and compound of Formula IID.

The reaction of the compound of Formula IV with the compound of Formula VI is carried out at an elevated temperature. In an embodiment, the reaction of the compound of Formula IV with the compound of Formula VI is carried out at a temperature in the range of 100-160 °C for a time-period in the range of 2 to 10 hours. In some embodiments, the reaction is carried out at 125-140 °C for 4-5 hours. If the reaction of the compound of Formula IV with the compound of Formula VI is carried out for more than 240 minutes, it results in increase in the extent of transamination with increase in viscosity.

An exemplary reaction scheme for obtaining the disclosed modified curing agent component (s) by reacting the compound of Formula IV with the compound of Formula VI is provided below:

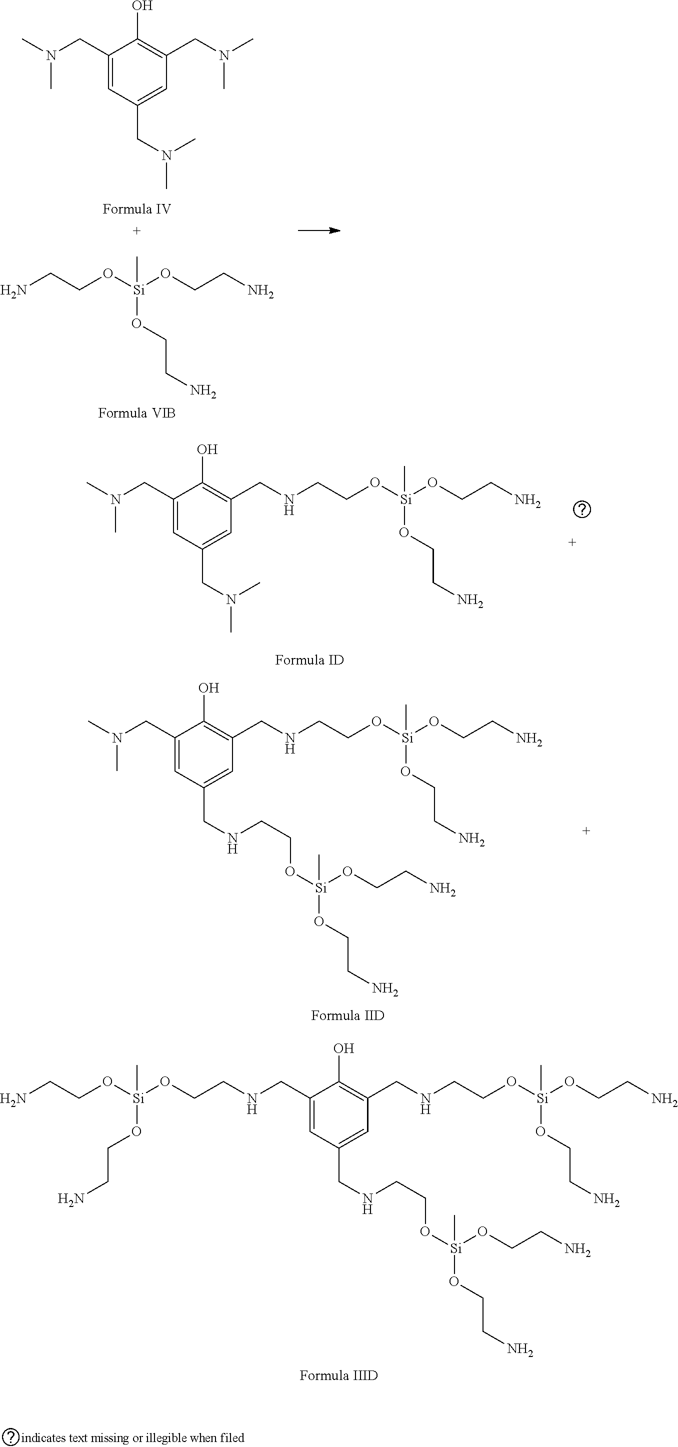

In the above scheme, Formula IV is Tris(dimethylaminomethyl)phenol.

### Formula IV+V+VI

In an embodiment, the compound of Formula IV is reacted with the mixture of the compound of Formula V and the compound of Formula VI in a molar ratio in the range of 1:2-2.8:0.2-1. In some embodiments, the compound of Formula IV is reacted with the mixture of the compound of Formula V and the compound of Formula VI in the molar ratio of 1:2.5:0.5.

The reaction of the compound of Formula IV with the mixture of the compound of Formula V and the compound of Formula VI is carried out at an elevated temperature. In an embodiment, the reaction of the compound of Formula IV with the mixture of the compound of Formula V and the compound of Formula VI is carried out at a reaction temperature in the range of 100-160 °C for a time-period in the range of 2 to hours. In some embodiments, the reaction is carried out at 120-130 °C for 5-6 hours. If the reaction of the compound of Formula IV with the compound of Formula VI is carried out for more than 240 mins, it results in increase in extent of transamination and increase in viscosity.

An exemplary reaction scheme for the reaction of Formula IV with the mixture of the compound of Formula V and the compound of Formula VI to obtain the modified curing agent component of the compound of Formula I, the compound of Formula II and the compound of Formula III is provided below.

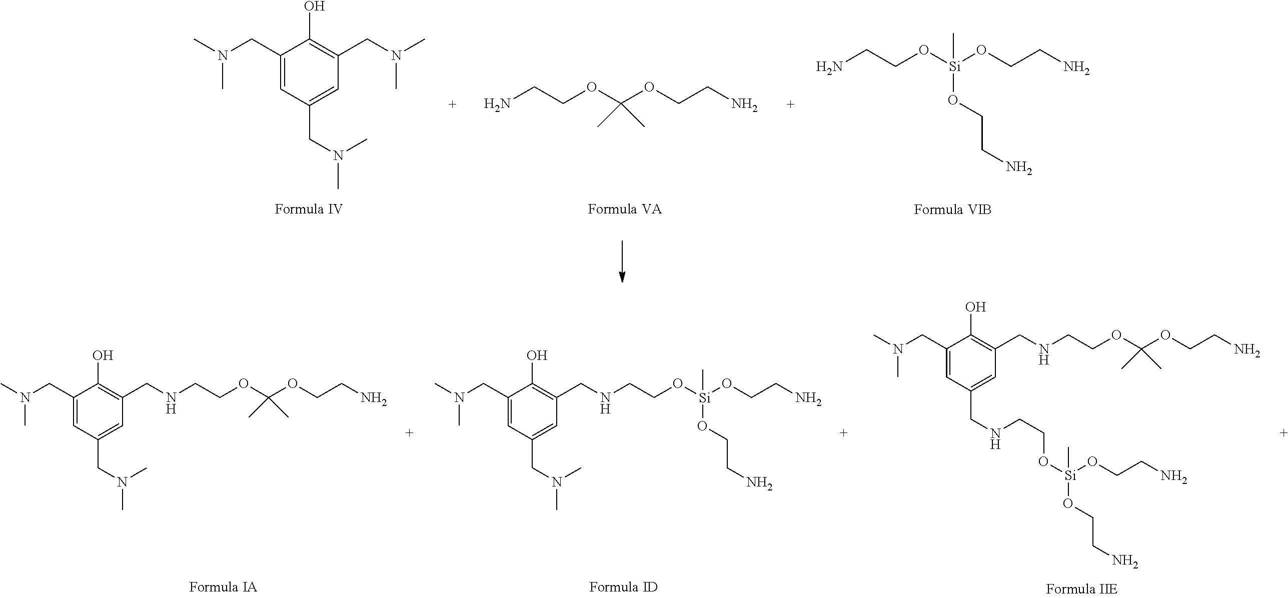

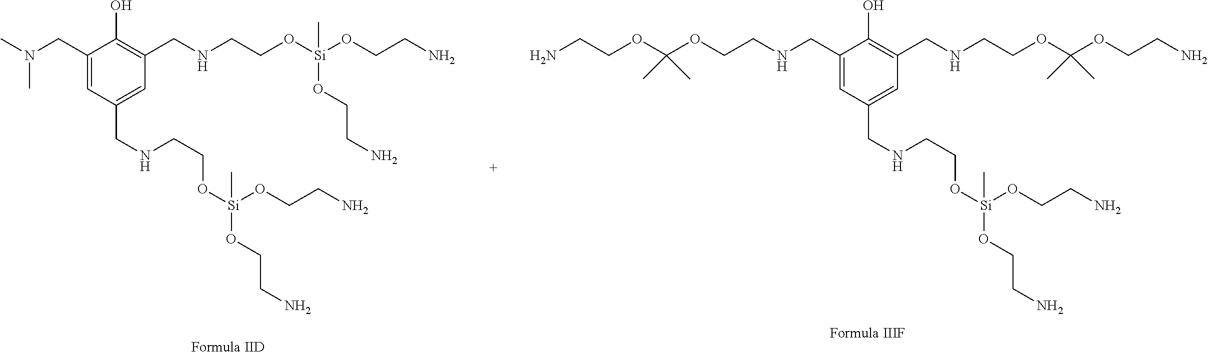 [SMILES: CN(C)Cc1cc(CNCCO[Si](C)(OCCN)OCCN)cc(CNCCO[Si](C)(OCCN)OCCN)c1O ; CC(C)(OCCN)OCCNCc1cc(CNCCO[Si](C)(OCCN)OCCN)cc(CNCCOC(C)(C)OCCN)c1O]

In the above scheme, Formula IV is Tris(dimethylaminomethyl)phenol.

In an embodiment, the modified curing agent component is also prepared by reacting the compound of Formula IV with the compound of Formula V and a compound selected from the group consisting of aliphatic amines, aromatic amines, cycloaliphatic amines and heterocyclic amines.

The modified curing agent component comprising the compound of Formula I, the compound of Formula II and the compound of formula III is obtained as a viscous liquid. In some embodiments, the viscous modified curing agent component may be subjected to further processing according to conventional methods or techniques known in the art. In an embodiment, the viscous curing agent component can be mixed or diluted with one or more commercially available curing agent or an accelerator or a solvent.

### Recyclable Epoxy Resin System

In another aspect, the present disclosure also relates to an epoxy resin system comprising at least one epoxy resin component and the modified curing agent component disclosed above.

In an embodiment, the epoxy resin system is recyclable. The modified curing agent component when cured with the epoxy resin component forms the cured epoxy resin thermoset that can be chemically broken down unlike a conventional epoxy resin system prepared by using the epoxy resin component and a conventional curing agent. The recyclable epoxy resin system is capable of softening in an acid solution at a temperature in the range of 60 to 100 °C. In some embodiments, the acid solution is an aqueous acetic acid solution. The recyclable epoxy resin system is capable of dissolution in these conditions because the formal linkage, the acetal linkage and the ketal linkage of the modified curing agent component is acid labile. These linkages undergo a bond cleavage in the acidic solution at an elevated temperature and hence allow the epoxy resin system to be recyclable.

The disclosed modified curing agent component comprising phenolic groups also result in faster drying of the recyclable epoxy resin system as compared to the conventional curing agent without adding the accelerator.

The modified curing agent component allows the epoxy resin component to cure rapidly, both at room temperature and at relatively lower ambient temperature. In an embodiment, the modified curing agent component cures the epoxy resin component faster at the temperature in the range of 0-10 °C as compared to the conventional curing agents. In some embodiments, the modified curing agent component cures the epoxy resin component at a temperature of about 5 °C and results in faster Shore-D hardness build up.

In embodiments, where the epoxy resin component is required to be cured at a lower temperature, the epoxy resin component is selected such that it is a liquid at the working temperature. In some embodiments, the epoxy resin component is a mixture of diglycidyl ether of bisphenol A and diglycidyl ether of bisphenol F. In another embodiment, the epoxy resin component is selected from the group consisting of diglycidyl ether of bisphenol A, diglycidyl ether of bisphenol F, diglycidyl ether of hydrogenated bisphenol A, glycidyl ethers of phenol-novalacs, triglcyidyl ether of p-aminophenol, and tetraglycidyl ether of methylene dianiline. In an embodiment, the epoxy resin system includes a diluent that allows the epoxy resin system to remain in liquid state even at a lower temperature. In an embodiment, the diluent is selected from a group consisting of phenol glycidyl ether, cresol glycidyl ether, diglycidyl ether of 1,4-butanediol, diglycidyl ether of 1,6-hexanediol, diglycidyl ether of neopentyl glycol, and monoglycidyl ether C12-C14 alcohol.

In accordance with an embodiment, the modified curing agent component and the epoxy resin component are added based on their Epoxy Equivalent Weight (EEW) and AHEW. The weight of the curing agent component (s) for 100 g of the epoxy resin component in the recyclable epoxy resin system may be calculated using below formula:

Weight of curing agent component=AHEW*100/Epoxy Equivalent Weight

In accordance with an embodiment, the viscosity of the epoxy resin component is in the range of 50-30000 cps. In accordance with an embodiment, the epoxy resin component has by products and impurities less than 10000 ppm.

In accordance with an embodiment, the recyclable epoxy resin system may further comprise additives. Said additives may be included as a separate component in addition to the modified curing agent component and the epoxy resin component. The additives include but are not limited to fibers, tougheners, flexibilizers, pigments, fillers, defoamers, wetting agents or combination thereof.

In accordance with an embodiment, the recyclable epoxy resin system has a gel time in the range of 30 to 1000 minutes. In some embodiments, the recyclable epoxy resin system has the gel time in the range of 50 to 500 minutes. In accordance with an embodiment, the recyclable epoxy resin system has a glass transition temperature in the range of 70 to 160 °C. In some embodiments, the recyclable epoxy resin system has the glass transition in the range of 85 to 130 °C.

In accordance with an embodiment, the recyclable epoxy resin system has a tensile strength in the range of 50 to 150 MPa. In some embodiments, the recyclable epoxy resin system has the tensile strength in the range of 60 to 100 MPa. In accordance with an embodiment, the recyclable epoxy resin system has an elongation at break in the range of 1% to 15%. In some embodiments, the recyclable epoxy resin system has the elongation at break in the range of 1% to 10%. In accordance with an embodiment, the recyclable epoxy resin system has a lap shear strength in the range of 1 to 10 MPa. In some embodiments, the recyclable epoxy resin system has the lap shear strength in the range of 2 to 5 MPa.

The following examples illustrate certain embodiments and aspects of the present invention and are not to be construed as limiting the scope thereof. All parts and percentages are on a weight basis unless otherwise stated.

### Example 1: Preparation of the Modified Curing Agent Component in Accordance with an Embodiment of the Present Disclosure

In a four necked reactor flask equipped with a temperature controller, a heating mantle, a water condenser, an overhead stirrer and a nitrogen inlet, 150 grams (0.565 mmole) of tris (dimethylaminomethyl)phenol and 275 grams (1.697 mmole) of the compound of Formula (VA) were charged to form a reaction mixture. The reaction mixture was heated at a temperature to 110-125 °C and DMA coming out of the water-cooled condenser was purged into a water vessel. The reaction mixture was maintained at 120 °C for 400 minutes under constant stirring. After 400 minutes of the reaction time, 60 grams of DMA was collected in a water vessel which corresponds to 79% transamination. 365 grams of a yellow viscous liquid containing the modified curing agent component was obtained.

Product characterization: Table 1 provides characteristics of the modified curing agent component obtained above.

TABLE 1
Characteristics of the modified curing agent component

| S. No. | Parameter | Characteristic |
|---|---|---|
| 1. | Amine value | 560 mgKOH/g |
| 2. | Viscosity cP@ 25 °C | 2780 |
| 3. | Amine Hydrogen Equivalent Weight (AHEW) | 68 |

### Example 2: Preparation of the Modified Curing Agent Component in Accordance with an Embodiment of the Present Disclosure

The experimental procedure of the example 1 was followed, except that the reaction mixture was refluxed for 360 minutes under constant stirring. 54.5 grams of DMA was collected in water vessel which corresponds to 71.5% transamination. 370.5 grams of a viscous yellow liquid containing the modified curing agent component was obtained.

Product characterization: Table 2 provides characteristics of the modified curing agent component obtained above.

TABLE 2
Characteristics of the modified curing agent component

| S. No. | Parameter | Characteristic | |
|---|---|---|---|
| 1. | Amine value | 567 | mgKOH/g |
| 2. | Viscosity @ 25 °C | 1980 | cP |

| | | |
|---|---|---|
| 3. | AHEW | 71 |

### Example 3: Preparation of the Modified Curing Agent Component in Accordance with an Embodiment of the Present Disclosure

The experimental procedure of the example 1 was followed, except that the reaction mixture was refluxed for 440 minutes under constant stirring. 73 grams of DMA was collected in a water vessel which corresponds to 96% transamination. 352 grams of a yellow viscous liquid containing the modified curing agent component was obtained.

Product characterization: Table 3 provides characteristics of the modified curing agent component obtained above.

TABLE 3
Characteristics of the modified curing agent component

| S. No. | Parameter | Characteristic | |
|---|---|---|---|
| 1. | Amine value | 561 | mgKOH/g |
| 2. | Viscosity @ 25 °C | 5369 | cP |

| | | |
|---|---|---|
| 3. | AHEW | 68 |

Observation: It was observed that when the reaction time was increased, the amount of formation of DMA increases which shows that the extent of transamination increases. As the extent of transamination increases, the compound of Formula I and the compound of Formula II are formed in greater proportions than the compound of Formula III.

### Example 4: Preparation of the Modified Curing Agent Component in Accordance with an Embodiment of the Present Disclosure

In a four necked reactor flask equipped with a temperature controller, a heating mantle, a water condenser, an overhead stirrer and a nitrogen inlet, 150 grams (0.565 mmole) of tris (dimethylaminomethyl)phenol and 275 grams (1.697 mmole) of the compound of Formula (VA) were charged to form a reaction mixture. The reaction mixture was heated at 110-125 °C and DMA coming out of the water-cooled condenser was purged into a water vessel. Thereafter, the reaction mixture was maintained at 120 °C for 330 minutes under constant stirring. After 330 minutes of the reaction time, 68 grams of DMA was collected in water vessel which corresponds to 89.5%% transamination. 329 grams of a yellow viscous liquid containing the modified curing agent component was obtained.

Product characterization: Table 4 provides characteristics of the modified curing agent component obtained above.

TABLE 4
Characteristics of the modified curing agent component

| S. No. | Parameter | Characteristic | |
|---|---|---|---|
| 1. | Amine value | 542 | mgKOH/g |
| 2. | Viscosity @ 25 °C | 9320 | cP |

| | | |
|---|---|---|
| 3. | AHEW | 71.6 |

### Example 5: Preparation of the Modified Curing Agent Component in Accordance with an Embodiment of the Present Disclosure

The experimental procedure followed in example 4 was followed, except that the reaction mixture was refluxed for 440 minutes. 56.5 grams of DMA was collected in a water vessel which corresponds to 74% transamination. 310 grams of a viscous yellow liquid containing the modified curing agent component was obtained.

Product characterization: Table 5 provides characteristics of the modified curing agent component obtained above.

TABLE 5
Characteristics of the modified curing agent component

| S. No. | Parameter | Characteristic | |
|---|---|---|---|
| 1. | Amine value | 572 | mgKOH/g |
| 2. | Viscosity @ 25 °C | 3280 | cP |

| | | |
|---|---|---|
| 3. | AHEW | 70 |

Observation: It was observed that when the reaction time was increased, the amount of formation of DMA increases which shows that the extent of transamination increases. As the extent of transamination increases, the compound of Formula I and the compound of Formula II are formed in greater proportions than the compound of Formula III.

### Example 6: Preparation of the Modified Curing Agent Component in Accordance with an Embodiment of the Present Disclosure

In a four necked reactor flask equipped with a temperature controller, a heating mantle, a water condenser, an overhead stirrer, and a nitrogen inlet, 100 grams (0.377 mmole) of tris (dimethylaminomethyl)phenol and 190 grams (0.989 mmole) of the compound of Formula (VB) were charged to form a reaction mixture. The reaction mixture was heated at 130-140 °C and DMA coming out of the water-cooled condenser was purged into a water vessel. Thereafter, the reaction mixture was maintained at 135 °C for 330 minutes under constant stirring. After 330 minutes of the reaction time, 40 grams of DMA was collected in water vessel which corresponds to 82% transamination. 250 grams of a yellow viscous liquid containing the modified curing agent component was obtained.

Product characterization: Table 6 provides characteristics of the modified curing agent component obtained above.

TABLE 6
Characteristics of the modified curing agent component

| S. No. | Parameter | Characteristic | |
|---|---|---|---|
| 1. | Amine value | 490 | mgKOH/g |
| 2. | Viscosity @ 25 °C | 7576 | cP |

| | | |
|---|---|---|
| 3. | AHEW | 81 |

### Example 7: Preparation of the Modified Curing Agent Component in Accordance with an Embodiment of the Present Disclosure

In a four necked reactor flask equipped with a temperature controller, a heating mantle, a water condenser, an overhead stirrer, and a nitrogen inlet, 100 grams (0.377 mmole) of tris (dimethylaminomethyl)phenol and 215 grams (1.119 mmole) of the compound of Formula VB were charged to form a reaction mixture. The reaction mixture was heated at 130-140 °C and DMA coming out of the water-cooled condenser was purged into a water vessel. Thereafter, the reaction mixture was maintained at 135 °C for 420 minutes under constant stirring. After 420 minutes of the reaction time, 47 grams of DMA was collected in water vessel which corresponds to 92% transamination. 268 grams of a yellow viscous liquid containing the modified curing agent component was obtained.

Product characterization: Table 7 provides characteristics of the modified curing agent component obtained above.

TABLE 7
Characteristics of the modified curing agent component

| S. No. | Parameter | Characteristic | |
|---|---|---|---|
| 1. | Amine value | 536 | mgKOH/g |
| 2. | Viscosity @ 25 °C | 15580 | cP |

| | | |
|---|---|---|
| 3. | AHEW | 77 |

### Example 8: Preparation of the Modified Curing Agent Component in Accordance with an Embodiment of the Present Disclosure

In a four necked reactor flask equipped with a temperature controller, a heating mantle, a water condenser, an overhead stirrer and a nitrogen inlet, 100 grams (0.377 mmole) of tris (dimethylaminomethyl)phenol and 213.6 grams (1.318 mmole) of the compound of Formula VA were charged to form a reaction mixture. This reaction mixture was heated at 110-125 °C. DMA coming out of the water-cooled condenser was purged into a water vessel. Thereafter, the reaction mixture was maintained at 125 °C for 270 minutes under constant stirring. After 270 minutes of the reaction time, grams of DMA was collected in water vessel which corresponds to 88% transamination. 169 grams of a viscous yellow liquid containing the modified curing agent component was obtained.

Product characterization: Table 8 provides characteristics of the modified curing agent component obtained above.

TABLE 8
Characteristics of the modified curing agent component

| S. No. | Parameter | Characteristic |
|---|---|---|
| 1. | Amine value mgKOH/g | 511 |
| 2. | Viscosity cP@ 25 °C | 2144 |
| 3. | AHEW | 64 |

### Example 9: Preparation of the Modified Curing Agent Component in Accordance with an Embodiment of the Present Disclosure

In a four necked reactor flask equipped with a temperature controller, a heating mantle, a water condenser, an overhead stirrer and a nitrogen inlet, 100 grams (0.377 mmole) of tris (dimethylaminomethyl)phenol and 244 grams (1.51 mmole) of the compound of Formula (VA) were charged to form a reaction mixture. The reaction mixture was heated at 110-125 °C and DMA coming out of the water-cooled condenser was purged into a water vessel. Thereafter, the reaction mixture was maintained at 120 °C for 360 minutes under constant stirring. After 360 minutes of the reaction time, 48 grams of DMA was collected in water vessel which corresponds to 94% transamination. 296 grams of a viscous yellow liquid containing the modified curing agent component was obtained.

Product characterization: Table 9 provides characteristics of the modified curing agent component obtained above.

TABLE 9
Characteristics of the modified curing agent component

| S. No. | Parameter | Characteristic |
|---|---|---|
| 1. | Amine value mgKOH/g | 539 |
| 2. | Viscosity cP@ 25 °C | 1588 |
| 3. | AHEW | 60 |

### Example 10: Preparation of the Modified Curing Agent Component in Accordance with an Embodiment of the Present Disclosure

In a four necked reactor flask equipped with a temperature controller, a heating mantle, a water condenser, an overhead stirrer and a nitrogen inlet, 100 grams (0.377 mmole) of tris (dimethylaminomethyl)phenol and 153 grams (0.944 mmole) of the compound of Formula (VA) were charged to form a reaction mixture. The reaction mixture was heated at 110-125 °C and DMA coming out of the water-cooled condenser was purged into a water vessel. Thereafter, the reaction mixture was maintained at 120 °C for 270 minutes under constant stirring. After 270 minutes of the reaction time, 38 grams of DMA was collected in water vessel which corresponds to 74.5% transamination. 217 grams of a yellow viscous liquid containing the modified curing agent component was obtained.

Product characterization: Table 10 provides characteristics of the modified curing agent component obtained above.

TABLE 10
Characteristics of the modified curing agent component

| S. No. | Parameter | Characteristic |
|---|---|---|
| 1. | Amine value mgKOH/g | 531 |
| 2. | Viscosity cP@ 25 °C | 3860 |
| 3. | AHEW | 60 |

### Example 11: Preparation of the Modified Curing Agent Component in Accordance with an Embodiment of the Present Disclosure

In a four necked reactor flask equipped with a temperature controller, a heating mantle, a water condenser, an overhead stirrer and a nitrogen inlet, 75 grams (0.285 mmole) of tris (dimethylaminomethyl)phenol and 91.6 grams (0.565 mmole) of the compound of Formula VA were charged to form a reaction mixture. The reaction mixture was heated at 110-125 °C and DMA coming out of the water-cooled condenser was purged into a water vessel. Thereafter, the reaction mixture was maintained at 120 °C for 50 minutes under constant stirring. Within 50 minutes of the reaction, the reaction mixture got gelled. This can be attributed to lower molar ratio of the compound of Formula VA which led to higher degree of polymerization leading to crosslinking/gelling.

### Example 12: Preparation of the Modified Curing Agent Component in Accordance with an Embodiment of the Present Disclosure

In a four necked reactor flask equipped with a temperature controller, a heating mantle, a water condenser, an overhead stirrer and a nitrogen inlet, 100 grams (0.377 mmole) of tris (dimethylaminomethyl)phenol and 248.7 grams (1.14 mmole) of the compound of Formula VD were charged to form a reaction mixture. This reaction mixture was heated at 130-140 °C for 360 minutes under constant stirring. The DMA coming out of the water-cooled condenser was purged into a water vessel. After 360 minutes of the reaction time, 30 grams of DMA was collected in a water vessel which corresponds to 58.5% transamination. 318.5 grams of a yellow viscous liquid containing the modified curing agent component was obtained.

Product characterization: Table 12 provides characteristics of the modified curing agent component obtained above.

TABLE 12
Characteristics of the modified curing agent component

| S. No. | Parameter | Characteristic |
|---|---|---|
| 1. | Amine value mgKOH/g | 470 |
| 2. | Viscosity cP@ 25 °C | 303 |
| 3. | AHEW | 82 |

### Example 13: Preparation of the Modified Curing Agent Component in Accordance with an Embodiment of the Present Disclosure

In a four necked reactor flask equipped with a temperature controller, a heating mantle, a water condenser, an overhead stirrer and a nitrogen inlet, 100 grams (0.377 mmole) of tris (dimethylaminomethyl)phenol and 153 grams (0.944 mmole) of the compound of Formula (VD) were charged to form a reaction mixture. The reaction mixture was heated at 140-150 °C for 360 minutes and DMA coming out of the water-cooled condenser was purged into a water vessel. After 360 minutes of the reaction time, 39 grams of DMA was collected in water vessel which corresponds to 75% transamination. 309 grams of a viscous yellow liquid containing the modified curing agent component was obtained.

Product characterization: Table 13 provides characteristics of the modified curing agent component obtained above.

TABLE 13
Characteristics of the modified curing agent component

| S. No. | Parameter | Characteristic |
|---|---|---|
| 1. | Amine value mgKOH/g | 417 |
| 2. | Viscosity cP@ 25 °C | 1330 |
| 3. | AHEW | 84.5 |

### Example 14: Preparation of the Modified Curing Agent Component in Accordance with an Embodiment of the Present Disclosure

In a four necked reactor flask equipped with a temperature controller, a heating mantle, a water condenser, an overhead stirrer and a nitrogen inlet, 100 grams (0.377 mmole) of tris (dimethylaminomethyl)phenol and 249 grams (1.141 mmole) of the compound of Formula (VE) were charged to form a reaction mixture. The reaction mixture was heated at 140-150 °C for 390 minutes and DMA which was coming out of the water-cooled condenser was purged into a water vessel. After 390 minutes of the reaction time, 35 grams of DMA was collected in water vessel which corresponds to 68.5% transamination. 314 grams of a yellow viscous liquid containing the modified curing agent component was obtained.

Product characterization: Table 14 provides characteristics of the modified curing agent component obtained above.

TABLE 14
Characteristics of the modified curing agent component

| S. No. | Parameter | Characteristic |
|---|---|---|
| 1. | Amine value mgKOH/g | 458 |
| 2. | Viscosity cP@ 25 °C | 2213 |
| 3. | AHEW | 83.75 |

### Example 15: Preparation of the Modified Curing Agent Component in Accordance with an Embodiment of the Present Disclosure

In a four necked reactor flask equipped with a temperature controller, a heating mantle, a water condenser, an overhead stirrer and a nitrogen inlet, 100 grams (0.377 mmole) of tris (dimethylaminomethyl)phenol and 152.6 grams (0.9419 mmole) of the compound of Formula (VA) and 57.9 gm (0.1892 mmole) of the compound of Formula (VIB) were charged to form a reaction mixture. The reaction mixture was heated at 120-130 °C for 360 minutes under continuous stirring. The DMA coming out of the water-cooled condenser was purged into a water vessel. After 360 minutes of the reaction time, 38.5 grams of DMA was collected in water vessel which corresponds to 75.5% transamination. 274 grams of a yellow viscous liquid containing the modified curing agent component was obtained.

Product characterization: Table 15 provides characteristics of the modified curing agent component obtained above.

TABLE 15
Characteristics of the modified curing agent component

| S. No. | Parameter | Characteristic |
|---|---|---|
| 1. | Amine value mgKOH/g | 531 |
| 2. | Viscosity cP@ 25 °C | 1910 |
| 3. | AHEW | 68 |

### Example 16: Preparation of the Modified Curing Agent Component in Accordance with an Embodiment of the Present Disclosure

In a four necked reactor flask equipped with a temperature controller, a heating mantle, a water condenser, an overhead stirrer and a nitrogen inlet, 100 grams (0.377 mmole) of tris (dimethylaminomethyl)phenol and 152.6 grams (0.9419 mmole) of the compound of Formula (VA) and 49.7 gm (0.1624 mmole) of the compound of Formula (VIC) were charged to form a reaction mixture. The reaction mixture was heated at 120-130 °C for 360 minutes. The DMA coming out of the water-cooled condenser was purged into a water vessel. After 360 minutes of the reaction time, 42 grams of DMA was collected in water vessel which corresponds to 82.5% transamination. 274 grams of a yellow viscous liquid containing the modified curing agent component was obtained.

Product characterization: Table 16 provides characteristics of the modified curing agent component obtained above.

TABLE 16
Characteristics of the modified curing agent component

| S. No. | Parameter | Characteristic |
|---|---|---|
| 1. | Amine value mgKOH/g | 561 |
| 2. | Viscosity cP@ 25 °C | 3320 |
| 3. | AHEW | 70 |

### Example 17: Preparation of the Recyclable Epoxy Resin System in Accordance with the Embodiment of the Present Disclosure

The recyclable epoxy resin systems were prepared by using the modified curing agent component prepared above and the epoxy resin component. Table 17 below provides the various recyclable epoxy resin systems that were prepared.

TABLE 17
Epoxy resin system formulation

| S. | | | |
|---|---|---|---|
| No. | Epoxy resin system | Curing agent | Epoxy resin component |
| 1. | Recyclable epoxy resin | Modified curing | Diglycidyl ether of |
| | system 1a | agent of example 1 | Bisphenol A |
| 2. | Recyclable epoxy resin | Modified curing | Diglycidyl ether of |
| | system 2a | agent example 2 | Bisphenol A |
| 3. | Recyclable epoxy resin | Modified curing | Diglycidyl ether of |
| | system 3a | agent example 3 | Bisphenol A |
| 4. | Recyclable epoxy resin | Modified curing | Mixture of diglycidyl ether |
| | system 1b | agent example 1 | of Bisphenol A and |
| | | | diglycidyl ether of |
| | | | Bisphenol F and diglycidyl |
| | | | ether of C12-C14 alcohol |
| 5. | Recyclable epoxy resin | Modified curing | Mixture of diglycidyl ether |
| | system 2b | agent example 2 | of Bisphenol A and |
| | | | diglycidyl ether of |
| | | | Bisphenol F and diglycidyl |
| | | | ether of C12-C14 alcohol |
| 6. | Recyclable epoxy resin | Modified curing | Mixture of diglycidyl ether |
| | system 3b | agent example 3 | of Bisphenol A and |
| | | | diglycidyl ether of |
| | | | Bisphenol F and diglycidyl |
| | | | ether of C12-C14 alcohol |
| 7. | Conventional epoxy | Compound of | Diglycidyl ether of |
| | resin system A | Formula VA. | bisphenol A |

Test conducted: The epoxy resin systems obtained above were tested for various processing properties.

### Gel Time:

### Gel Time at 25 °C:

The prepared recyclable epoxy resin systems 1a, 2a, and 3a were evaluated for gel time at 25 °C by using Gelnorm Gel Timer GT-SP. The total batch size for the experiment conducted was 100 grams. The stamper was dipped inside the cup containing the recyclable epoxy resin systems 1a, 2a and 3a and the instrument was set in operation. The timer was put on and after the epoxy resin systems has completely gelled, the timer reading was noted as the gel time. Table 18 provides the gel time of the recyclable epoxy resin systems 1a, 2a, and 3a and conventional epoxy system A at °C.

TABLE 18
Gel time of the recyclable epoxy resin systems 1a, 2a,
and 3a and conventional epoxy resin system A at 25 °C.

| | Conventional | Recyclable | Recyclable | Recyclable |
|---|---|---|---|---|
| Processing | epoxy resin | epoxy resin | epoxy resin | epoxy resin |
| Properties | system A | system 1a | system 2a | system 3a |
| Gel time, 100 | 85 | 83 | 82 | 77 |
| gm mix @ | | | | |
| 25 °C (in min) | | | | |

Observations: The results showed that there was not much difference in the gel time of the recyclable epoxy resin systems 1a, 2a, and 3a and the conventional epoxy resin system A at 25 °C, indicating that the modified curing agent component has similar reactivity as the conventional curing agent.

### Gel Time at 5-7 °C

The recyclable epoxy resin systems 1b, 2b, and 3b and the conventional epoxy resin system A prepared in table 17 above were evaluated for gel time at 5-7 °C by using the Gelnorm Gel Timer GT-SP. Table 19 provides the gel time of the recyclable epoxy resin systems 1b, 2b, and 3b and conventional epoxy resin system A at 7 °C.

TABLE 19
Gel time of the recyclable epoxy resin systems 1b, 2b,
and 3b and conventional epoxy resin system A at 5-7 °C.

| | Conventional | Recyclable | Recyclable | Recyclable |
|---|---|---|---|---|
| Processing | epoxy resin | epoxy resin | epoxy resin | epoxy resin |
| Properties | system A | system 1b | system 2b | system 3b |
| Gel time, | 19 | 10.5 | 10.5 | 10 |
| 100 gm | | | | |
| mix (in | | | | |
| hours) | | | | |

Observations: The result showed that at low temperature of 5-7 °C, the recyclable epoxy resin systems 1b, 2b and 3b showed faster gel times as compared to the conventional epoxy resin system A. This indicates that the reactivity of the modified curing agent component was better at low temperature than that of the conventional curing agent.

Processing properties: The recyclable epoxy resin systems 1a, 2a, and 3a was cured at 80 °C for 4 hours in a closed system and were tested for glass transition temperature, tensile strength, elongation at break and lap shear strength.

### Glass Transition Temperature (Tg):

Tg was tested using a Differential Scanning Calorimetry (DSC) as per ASTM D 3418. The results are provided in table 20 below.

### Tensile Strength and % Elongation:

The prepared recyclable epoxy resin systems 1a, 2a and 3a and the conventional epoxy resin system A were casted in a dumbbell shaped mould having effective length of 50 mm and width of 10 mm to prepare a dumbbell shaped specimen. The prepared specimens were cured at a temperature of 80 °C for 4 hours for full curing. The tensile strength and % elongation was tested according to ASTM D638. The results are provided in table 20 below.

### Lap Shear Strength:

The prepared recyclable epoxy resin systems 1a, 2a and 3a and the conventional epoxy resin system A were used as an adhesive on 25×50 mm aluminium plates. 25×25 mm area on one side of the plate was first coated with the prepared epoxy resin systems. The second aluminium plate was sticked together as per the standard to prepare a testing specimen as per ASTM D1002 standard. The specimens were cured at a temperature of 80 °C for 4 hours for full curing. The lap shear strength of the prepared specimens was tested according to ASTM D1002. The results are provided in table 20 below.

Table 20 provides the processing properties of the recyclable epoxy resin systems prepared 1a, 2a, 3a and the conventional epoxy resin system A.

TABLE 20
Processing Properties

| | Conventional | Recyclable | Recyclable | Recyclable |
|---|---|---|---|---|
| Processing | epoxy resin | epoxy resin | epoxy resin | epoxy resin |
| Properties | system A | system 1a | system 2a | system 3a |
| Glass | 93.2 | 103.8 | 102.3 | 104.1 |
| transition | | | | |
| temperature | | | | |
| (Tg, in °C) | | | | |
| Tensile | 66.6 | 67.3 | 69.2 | 72.9 |
| Strength (MPa) | | | | |
| Elongation at | 6.2 | 6.2 | 5.7 | 6.2 |
| break (%) | | | | |
| Lap Shear | 3.47 | 2.98 | 3.5 | 2.93 |
| Strength (MPa) | | | | |

Observations: The recyclable epoxy resin systems 1a, 2a, and 3a also showed an increase in the glass transition temperature as compared to the conventional epoxy resin system A. The increase in the glass transition temperature was primarily because of the higher functionality in the modified curing agent component, which led to dense crosslinking, which in turn restricts molecular movements, resulting in higher Tg.

The tensile strength also showed a gradual increase for the recyclable epoxy resin systems 1a, 2a, and 3a. This was the result of the higher crosslinking or denser three-dimensional network created by the multi-functional nature of the modified curing agent component of the present disclosure.

### Example 18: Preparation of the Recyclable Epoxy Resin System in Accordance with an Embodiment of the Present Disclosure

The recyclable epoxy resin system 1c and the conventional epoxy resin system B were prepared by using the curing agent and the epoxy resin component as specified in Table 21 below.

TABLE 21
Epoxy resin system formulation

| | | | Epoxy resin |
|---|---|---|---|
| S. No. | Epoxy resin system | Curing agent | component |
| 1. | Recyclable epoxy | Modified curing agent | Diglycidyl ether of |
| | resin system 1c | component of example 7 | Bisphenol A |
| 2. | Conventional epoxy | Compound of Formula | Diglycidyl ether of |
| | resin system B | VB | Bisphenol A |

The modified curing agent component obtained in example 7 was cured with diglycidyl ether of bisphenol A at 80 °C for 4 hours in a closed system to form the recyclable epoxy resin system 1c.

The conventional epoxy resin system B was prepared by curing the diglycidyl ether of bisphenol A and the compound of Formula VB at 80 °C for 4 hours in a closed system.

The prepared recyclable epoxy resin system 1c and the conventional epoxy resin system B were tested for gel time, glass transition temperature, tensile strength, elongation at break and lap shear strength. Table 22 below provides the performance properties of the recyclable epoxy resin system 1c and the conventional epoxy resin system B.

TABLE 22
Processing Properties

| | Conventional epoxy | Recyclable epoxy |
|---|---|---|
| Processing Properties | resin system B | resin system 1c |
| Gel time, 100 gm mix @ | 505 | 410 |
| 25 °C (in min) | | |
| Glass transition | 110.9 | 120.8 |
| temperature (Tg) | | |
| Tensile Strength (MPa) | 65.2 | 92.5 |
| Elongation at break (%) | 3.8 | 6.1 |
| Lap Shear Strength (MPa) | 2.54 | 3.89 |

Observation: The results showed a significant improvement in gel time for the recyclable epoxy resin system 1c as compared to the conventional epoxy resin system B, indicating faster reactivity even at room temperature (25 °C). The tensile strength and lap shear strength of the recyclable epoxy resin system 1c also improved against the conventional epoxy resin system B, indicating faster and complete curing of the epoxy resin systems, leading to better strength.

### Example 19: Preparation of the Coating Compositions

The recyclable epoxy resin systems 1b, 2b and 3b were prepared by curing the epoxy resin component and the curing agent as specified in table 17 at 5-8 °C. The prepared recyclable epoxy resin systems 1b, 2b and 3b were used to prepare the coating films 1, 2, and 3.

The conventional epoxy resin system C was prepared by curing the diglycidyl ether of bisphenol A and the compound of Formula VA. The conventional epoxy resin system C is used to prepare the conventional coating film.

For the evaluation of the drying performance, thin films of 400 μthickness were coated on a glass plate by using the recyclable epoxy resin systems 1b, 2b and 3b and the conventional epoxy resin system C. Thereafter, the films were casted for Shore D Hardness measurement as per ASTM D 2240 and cured at 5-8 °C.

The performance properties of the coating films are tabulated in Table 23.

TABLE 23
Performance properties of the coating film

| Performance | | Conventional | Coating | Coating | Coating film |
|---|---|---|---|---|---|
| Properties | Day | coating film | film 1 | film 2 | of example 3 |
| Shore D | 1 | * | * | * | * |
| Hardness | 2 | * | 11 | 10 | 10 |
| | 3 | 12 | 69 | 63 | 65 |
| | 4 | 58 | 76 | 77 | 76 |
| | 7 | 73 | 81 | 81 | 79 |
| Film drying | 1 | Tacky | Tacky | Tacky | Tacky |
| | 2 | Tacky | Tacky | Tacky | Tacky |
| | 3 | Tacky | Cured | Cured | Cured |
| | 4 | Tacky | — | — | — |
| | 7 | Tacky | — | — | — |

*Not possible to test as the samples were not sufficiently cured
—completely cured/dried

Observation: The shore D hardness measurement indicated that the coating films prepared by using the recyclable epoxy resin systems 1b, 2b, and 3b led to faster drying at lower temperatures, which is evident from the film drying results. The faster drying was due to the faster reactivity of the modified curing agent component as compared to the conventional curing agent.

### Example 21: Recycling of Epoxy Thermoset

A cured specimen (similar to dumbbell shaped specimen) prepared from the recyclable epoxy resin system of the present disclosure was kept in 25% of an aqueous acetic acid solution at 100 °C for 1 hour. Within 30 minutes of immersion of the specimen in the acidic solution, the specimen started to soften and disintegrates. Within 1 hour, the specimen completely disintegrates, and is recovered as a thermoplastic polymer post purification.

### INDUSTRIAL APPLICABILITY

The modified curing agent component disclosed herein allows to produce epoxy resin system having reworkable and recyclable properties. In addition, the modified curing agent component allows faster curing at low temperature, fast strength development, and higher cross-link density.

The modified curing agent component cures rapidly with the epoxy resin component both at room temperature and at low temperature to form the recyclable epoxy resin system. The curing of the modified curing agent component with the epoxy resin component at low temperatures minimizes internal stresses within the recyclable epoxy resin system, leading to improved dimensional stability and performance.

The recyclable epoxy resin system obtained by using the modified curing agent component possesses desirable processing and performance properties suitable for a wide range of applications including high-tech fields for examples microelectronics, transportation, aerospace industries and composite applications. Additionally, the recyclable epoxy resin system has suitable characteristic that make them amenable for use in standard thermosetting composite manufacturing techniques such as wet lay-up, filament winding, vacuum infusion, compression molding, resin transfer molding. These composite materials prepared from recyclable epoxy resin system have excellent mechanical properties that make them useful for different composite applications. Moreover, the composite materials that are obtained by using the recyclable epoxy resin system can also be degraded under specific conditions, leading to the separation and recovery of both the reinforcing fiber and the epoxy resin component and the modified curing agent component. These composite materials can be recycled precisely because the epoxy matrix of a fabricated composite is derived from reworkable modified curing agent components.

We claim:

1. A modified curing agent component for an epoxy resin system, the modified curing agent component comprising of:
a compound of Formula I represented by: 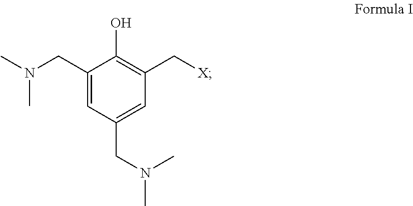
wherein each of X is independently selected from: 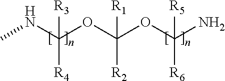
wherein:
n is 1-5; and
each of R₁, R₂, R₃, R₄, R₅ and R₆ is selected from the group consisting of H, CH₃, C₂H₅ and a combination thereof;
or 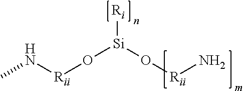
wherein
n is 0-2;
m is 1-3;
Ri is independently methyl, or ethyl; and
each of Rii is independently selected from the group consisting of ethylene, propylene, isopropylene, butylene, isobutylene and a combination thereof;
a compound of Formula II represented by: 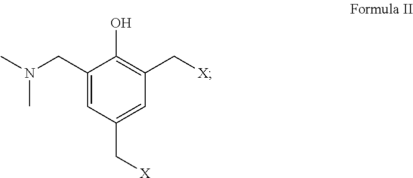
wherein each of X is independently selected from: 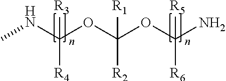
wherein:
n is 1-5; and
each of R1, R2, R3, R4, R5 and R6 is independently selected from the group consisting of H, CH₃, C₂H₅ and a combination thereof;
or 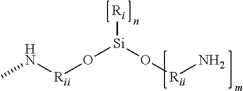
wherein
n is 0-2;
m is 1-3;
Ri is independently methyl, or ethyl; and
each of Rii is independently selected from the group consisting of ethylene, propylene, isopropylene, butylene, isobutylene and a combination thereof; and
a compound of Formula III, represented by: 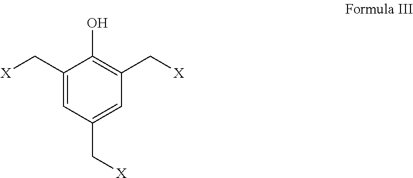
wherein each of X is independently selected from: 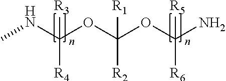
wherein:
n is 1-5; and
each of R1, R2, R3, R4, R5 and R6 is independently selected from the group consisting of H, CH₃, C₂H₅ and a combination thereof;
or 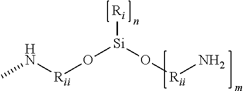
wherein
n is 0-2;
m is 1-3;
Ri is independently selected from methyl, or ethyl; and
each of Rii is independently selected from the group consisting of ethylene, propylene, isopropylene, butylene, isobutylene and a combination thereof.

2. The modified curing agent component as claimed in claim 1, wherein the compound of Formula I is selected from the group consisting of a compound of Formula IA, a compound of Formula IB, a compound of Formula IC, and a compound of Formula ID represented by structures:

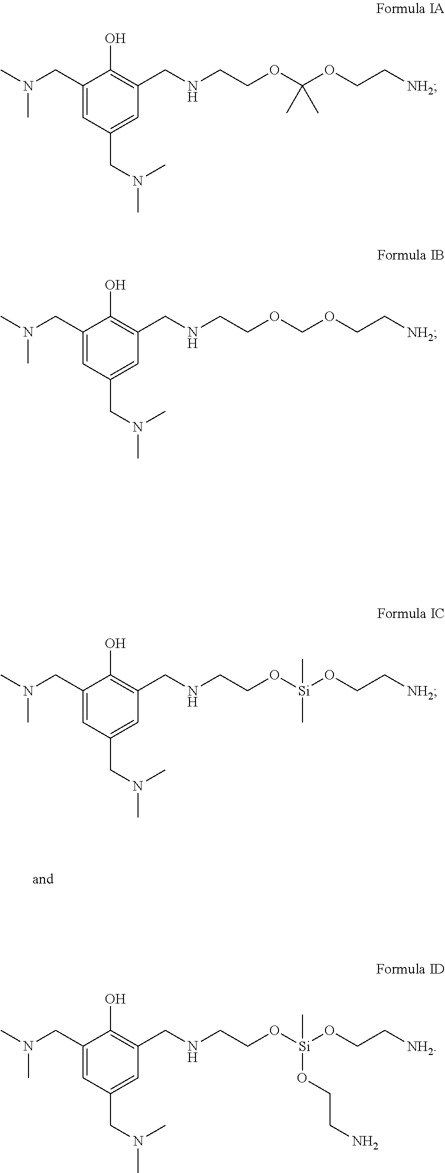 [SMILES: CN(C)Cc1cc(CNCCOC(C)(C)OCCN)c(O)c(CN(C)C)c1 ; CN(C)Cc1cc(CNCCOCOCCN)c(O)c(CN(C)C)c1 ; CN(C)Cc1cc(CNCCO[Si](C)(C)OCCN)c(O)c(CN(C)C)c1 ; CN(C)Cc1cc(CNCCO[Si](C)(OCCN)OCCN)c(O)c(CN(C)C)c1]

3. The modified curing agent component as claimed in claim 1, wherein the compound of Formula II is selected from the group consisting of a compound of Formula IIA, a compound of Formula IIB, a compound of Formula IIC, a compound of Formula IID and a compound of Formula TIE represented by structures:

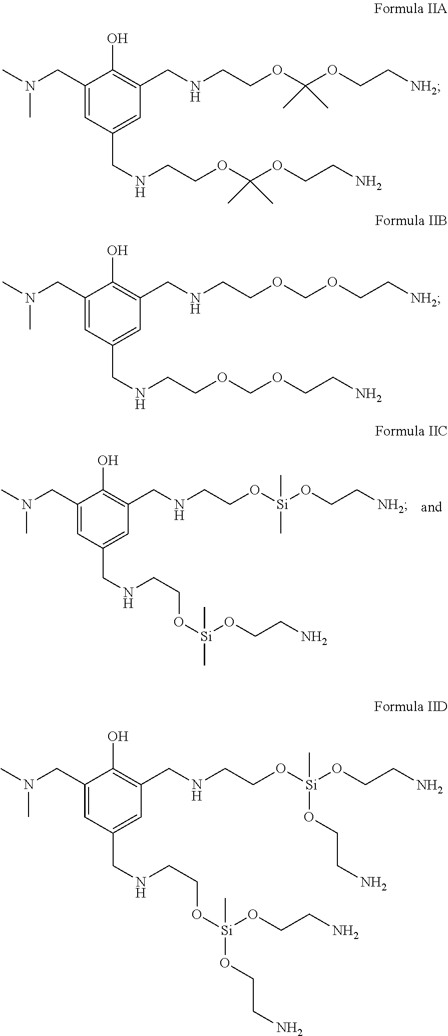 [SMILES: CN(C)Cc1cc(CNCCOC(C)(C)OCCN)cc(CNCCOC(C)(C)OCCN)c1O ; CN(C)Cc1cc(CNCCOCOCCN)cc(CNCCOCOCCN)c1O ; CN(C)Cc1cc(CNCCO[Si](C)(C)OCCN)cc(CNCCO[Si](C)(C)OCCN)c1O ; CN(C)Cc1cc(CNCCO[Si](C)(OCCN)OCCN)cc(CNCCO[Si](C)(OCCN)OCCN)c1O]

4. The modified curing agent component as claimed in claim 1, wherein the compound of Formula III is selected from the group consisting of a compound of Formula IIIA, a compound of Formula IIIB, a compound of Formula IIIC, a compound of Formula HID, a compound of Formula IIIE and a compound of Formula IIIF represented by structures:

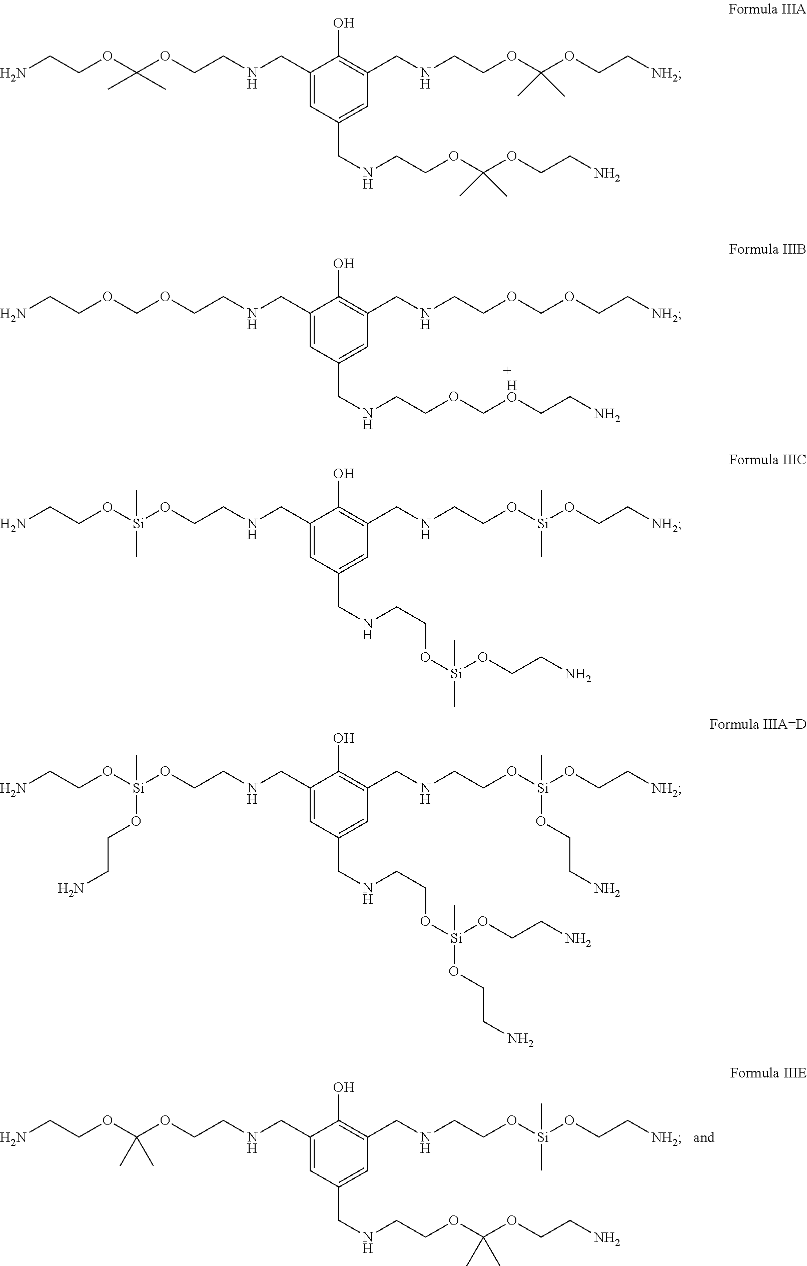

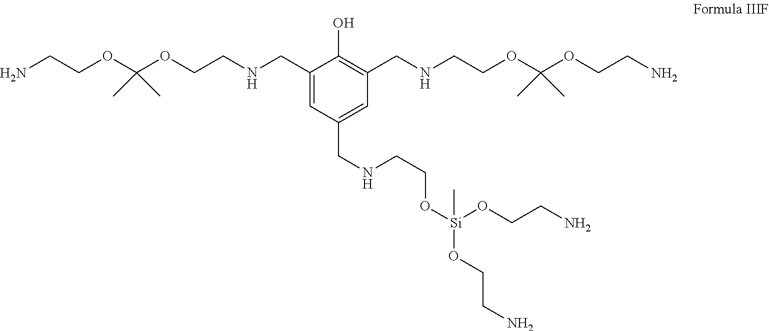 [SMILES: CC(C)(OCCN)OCCNCc1cc(CNCCO[Si](C)(OCCN)OCCN)cc(CNCCOC(C)(C)OCCN)c1O]

5. The process for preparing a modified curing agent component, said process comprising reacting a compound of Formula IV with a compound selected from the group consisting of a compound of Formula V, a compound of Formula VI and a combination thereof, wherein the compound of Formula IV, the compound of Formula V and the compound of Formula VI are represented by the structures: 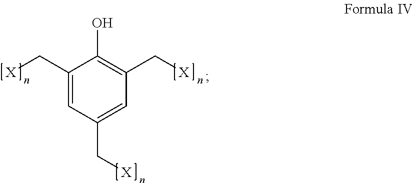
wherein
n is 0-1;
X is either 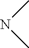
or OH; 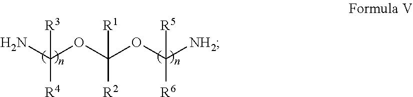
wherein
n is 1-5;
each R₁ to R₆ is independently selected from the group consisting of H, CH₃, and C₂H₅;
and 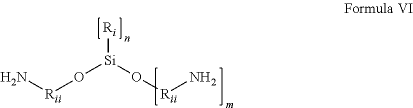
wherein
n is 0-2;
m is 1-3;
Rᵢ is independently selected from methyl, or ethyl;
each of Rᵢᵢ is independently selected from the group consisting of ethylene, propylene, isopropylene, butylene, isobutylene and a combination thereof;
to obtain the modified curing agent component comprising:
a compound of Formula I, represented by: 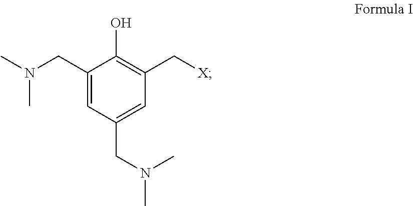
wherein each of X is independently selected from: 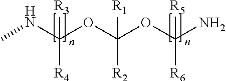
wherein:
n is 1-5; and
each of R1, R2, R3, R4, R5 and R6 is selected from the group consisting of H, CH3, C₂H₅ and a combination thereof;
or 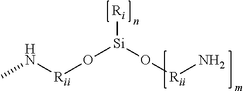
wherein
n is 0-2;
m is 1-3;
Ri is independently methyl, or ethyl; and
each of Rii is independently selected from the group consisting of ethylene, propylene, isopropylene, butylene, isobutylene and a combination thereof.
a compound of Formula II represented by: 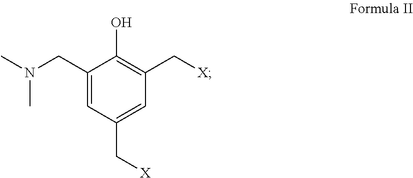
wherein each of X is independently selected from: 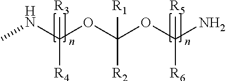
wherein:
n is 1-5; and
each of R1, R2, R3, R4, R5 and R6 is selected from the group consisting of H, CH3, C2H5 and a combination thereof;
or 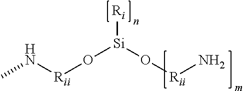
wherein:
n is 0-2;
m is 1-3;
Ri is independently methyl or ethyl; and
each of Rii is independently selected from the group consisting of ethylene, propylene, isopropylene, butylene, isobutylene and a combination thereof;
a compound of Formula III represented by: 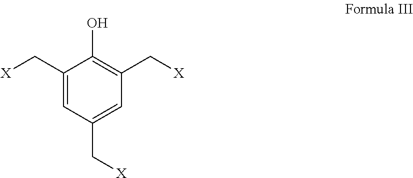
wherein each of X is independently selected from: 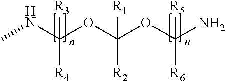
wherein
n is 1-5; and
each of R1, R2, R3, R4, R5 and R6 is selected from the group consisting of H, CH3, C2H5 and a combination thereof;
or 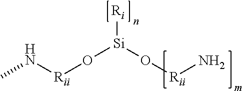
wherein
n is 0-2;
m is 1-3;
Ri is independently methyl, or ethyl; and
each of Rii is independently selected from the group consisting of ethylene, propylene, isopropylene, butylene, isobutylene and a combination thereof.

6. The process as claimed in claim 5, wherein the compound of Formula IV is reacted with the compound of Formula V in a molar ratio in the range of 1:2 to 1:6.

7. The process as claimed in claim 5, wherein the compound of Formula IV is reacted with the compound of Formula VI in a molar ratio in the range of 1:2.5 to 1:4.

8. The process as claimed in claim 5, wherein the compound of Formula IV is reacted with the combination of the compound of Formula V and the compound of Formula VI in a molar ratio in the range of 1:2-2.8:0.2-1.

9. The process as claimed in claim 5, wherein the reaction of the compound of Formula IV with the compound selected from the group consisting of the compound of Formula V, the compound of Formula VI and the combination thereof is carried out at a temperature in the range of 100-160 °C for a time-period in the range of 2 to 10 hours.

10. The process as claimed in claim 5, wherein the compound of Formula IV is selected from the group consisting of Tris(dimethylaminomethyl)phenol, Bis[(dimethylamino)methyl]phenol, ((dimethylamino)methyl)phenol, and a combination thereof.

11. The process as claimed in claim 5, wherein the compound of Formula IV is selected from the group consisting of 2,4-bihydroxymethyl phenol, 2,6-bishydroxymethyl phenol and a combination thereof.

12. The process as claimed in claim 5, wherein the compound of Formula V is selected from a group consisting of a compound of Formula VA, a compound of Formula VB, a compound of Formula VC, a compound of Formula VD, and a compound of Formula VE, represented by:

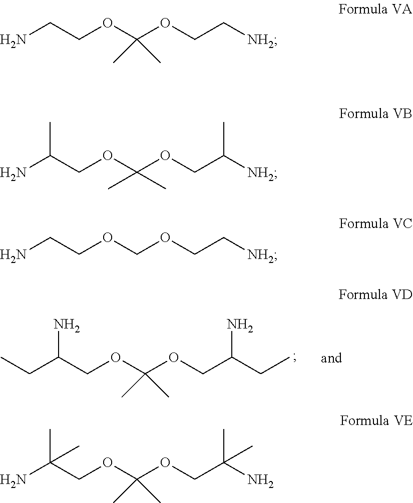

13. The process as claimed in claim 5, wherein the compound of Formula VI is selected from the group consisting of a compound of Formula VIA, a compound of Formula VIB, a compound of Formula VIC, a compound of Formula VID, and a compound of Formula VIE represented by: 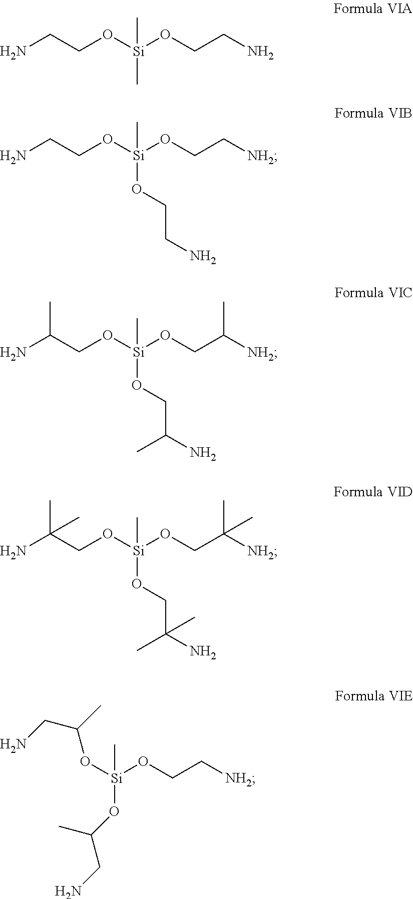
and

14. A recyclable epoxy resin system comprising at least one epoxy resin component and a modified curing agent component comprising of:
a compound of Formula I represented by: 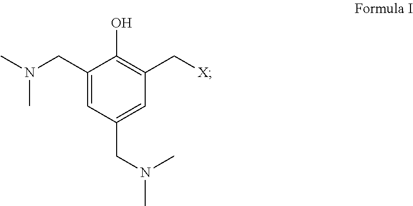
wherein each of X is independently selected from: 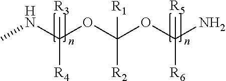
wherein:
n is 1-5; and
each of R1, R₂, R₃, R₄, R₅ and R₆ is selected from the group consisting of H, CH₃, C₂H₅ and a combination thereof;
or 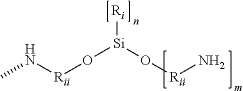
wherein:
n is 0-2;
m is 1-3;
Ri is independently methyl, or ethyl; and
each of Rii is independently selected from the group consisting of ethylene, propylene, isopropylene, butylene, isobutylene and a combination thereof;
a compound of Formula II represented by: 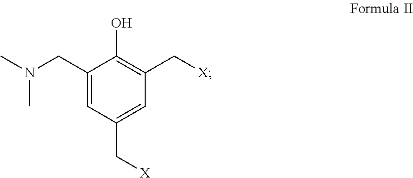
wherein each of X is independently selected from: 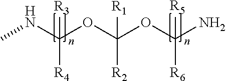
wherein:
n is 1-5; and
each of R1, R2, R3, R4, R5 and R6 is independently selected from the group consisting of H, CH3, C2H5 and a combination thereof;
or 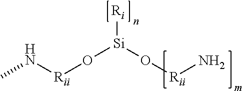
wherein
n is 0-2;
m is 1-3;
Ri is independently methyl, or ethyl; and
each of Rii is independently selected from the group consisting of ethylene, propylene, isopropylene, butylene, isobutylene and a combination thereof; and
a compound of Formula III, represented by: 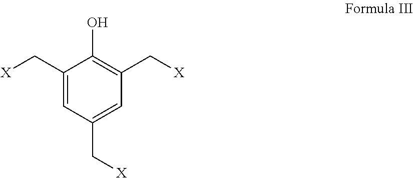
wherein each of X is independently selected from: 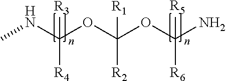
wherein n is 1-5; and
each of R1, R2, R3, R4, R5 and R6 is independently selected from the group consisting of H, CH3, C2H5 and a combination thereof;
or 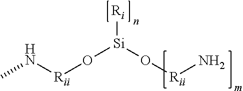
wherein
n is 0-2;
m is 1-3;
Ri is independently selected from methyl, or ethyl; and
each of Rii is independently selected from the group consisting of ethylene, propylene, isopropylene, butylene, isobutylene and a combination thereof;
wherein the recyclable epoxy resin system has a curing temperature in the range of 0 °C-10 °C.

15. The recyclable epoxy resin system as claimed in claim 14, wherein the epoxy resin component is selected from the group consisting of diglycidyl ether of bisphenol A, diglycidyl ether of bisphenol F, a mixture of diglycidyl ether of bisphenol A and bisphenol F, diglycidyl ether of hydrogenated bisphenol A, glycidyl ethers of phenol-novalacs, triglycidyl ether of p-aminophenol, and tetraglycidyl ether of methylene dianiline and a combination thereof.

16. The recyclable epoxy resin system as claimed in claim 14, further comprises a diluent selected from the group consisting of phenol glycidyl ether, cresol glycidyl ether, diglycidyl ether of 1,4-butanediol, diglycidyl ether of 1,6-hexanediol, diglycidyl ether of neopentyl glycol, and monoglycidyl ether C12-C14 alcohol.
